(19) United States
(12) Patent Application Publication
(10) Pub. No.: US 2026/0207810 A1
(43) Pub. Date: Jul. 23, 2026

(54) Customizable Medical Waste Processor

(71) Applicant: Spectrum Medical Lending, LLC, Chicago, IL (US)

(72) Inventors: Christopher Ryan Spock, Watertown, MA (US); Devin Michael Crowley, Berlin, MA (US); Antonio Batalha, JR., Chicopee, MA (US)

(73) Assignee: Spectrum Medical Lending, LLC, Chicago, IL (US)

(21) Appl. No.: 19/355,414

(22) Filed: Oct. 10, 2025

Related U.S. Application Data
(60) Provisional application No. 63/711,022, filed on Oct. 23, 2024.

(51) Int. Cl. A61L 11/00; A61L 2/07

(52) U.S. Cl. CPC A61L 11/00; A61L 2/07; A61L 2202/11; A61L 2202/121; A61L 2202/16

(57) ABSTRACT

A medical waste sterilization and processing device and method includes a first compartment having a top lid, central chamber, and bottom lid; and a steam generator supplying pressurized steam to the compartment. The top lid is openable to receive medical waste; and the bottom lid is sealable to support the medical waste. A press plate is configured for alternate upstream and downstream movement within the central chamber by an actuator, and includes a resilient flange extending transversely along an outer edge thereof to engage and wipe the inner surface of the central chamber during the upstream and downstream movement. A grinder disposed downstream of the first sealable compartment receives the treated waste pushed out of the first compartment by the press plate when the bottom lid is opened, and grinds/shreds the treated waste to produce shredded waste. A second compartment downstream of the grinder receives the shredded waste for removal.

### RELATED APPLICATION

This application is related to U.S. Pat. No. 9,676,012 entitled Devices for Treating Medical Waste and Methods of Their Use, issued on Jun. 13, 2017, and claims the benefit of U.S. Provisional Patent Application Ser. No. 63/711,022, entitled Customizable Waste Types—Medical Waste Processor with Software 2.0, filed on Oct. 23, 2024, the contents both of which are incorporated herein by reference in their entirety for all purposes.

### TECHNICAL FIELD

This invention relates to the treatment of medical waste, and more particularly to devices and methods for treating medical waste.

### BACKGROUND

### Background Information

Conventional devices and methods for treating medical waste include collecting the waste, storing the waste for a period of time, transporting the waste to a waste treatment center and disposing of the waste by such methods as incineration. Rules relating to the handling of waste differ according to local laws. As a result, medical waste can fester during storage and can be dangerous to handle when collecting, transporting and disposing of such waste. In addition, the movement of medical waste from its point of origin to a storage area exposes handlers, facilities and the environment to potential contamination.

In response to these issues, devices and methods have been described that treat medical waste at the facility in which it is created. The waste is collected and, in some cases stored prior to being placed in the device and sterilized using a number of techniques, either individually or collectively, including chemical treatment, steam autoclaving, microwaving, and ozonation. The treated waste is then collected, packaged, transported and discarded, as the waste is no longer considered, by law, a biological hazard. These conventional devices tend to be relatively large, expensive, permanent fixtures which still require collection of the medical waste from the point of origin by personnel who may not be fully trained in the science of infectious waste exposure or emergency procedures needed in the case of a spill or other accident. The operation and maintenance of these devices may also require extensive training.

In response to these issues, devices and methods have been described which are smaller and more suitable for being located on hospital floors, in emergency rooms, or in treatment rooms, i.e., places where medical waste is generated. Medical waste is often collected in red bags, or in sharps containers, until sufficiently full and then transferred to a treatment area where it is treated to render the waste biologically benign. Many of these devices, however, need to be installed using special plumbing and/or special electrical power connections. Once installed, these devices are generally intended to remain stationary, due to the difficulty and relative expense of reinstallation. Moreover, many conventional devices are limited as to the types of waste they are able to process, being unable to process relatively low melting point plastics and/or brittle materials such as glass slides that tend to clog or otherwise damage system components. Thus, there is an ongoing need for devices and methods for the treatment of medical waste at the point of origin that are sized to fit readily within a medical environment and are readily moveable and self-contained such that no special installation is required, and which are able to process a wide variety of medical waste to avoid having to segregate the waste prior to treatment.

### SUMMARY

In one aspect of the present invention, a medical waste sterilization and processing device includes a first sealable compartment having a top lid being selectively moveable between open and sealed positions, a central chamber, and a bottom lid being selectively moveable between open and sealed positions; and a steam generating component for supplying steam at elevated temperatures and pressures to the compartment. The top lid is moveable to its open position to receive medical waste into the central chamber; and the bottom lid is moveable to its sealed position to support the medical waste within the central chamber. The compartment is configured to maintain elevated steam temperatures and pressures therein when sealed. A press plate sized and shaped to form a sliding fit with an inner surface of the central chamber is configured for alternate upstream and downstream movement within the central chamber by an actuator including a scissor jack, piston and/or an inflation bladder. The press plate has a resilient flange extending transversely along an outer edge thereof, to engage and wipe the inner surface of the central chamber during the upstream and downstream movement. A grinder is disposed downstream of the first sealable compartment to receive the treated waste pushed out of the first compartment by the downstream movement of the press plate when the bottom lid is opened, and to grind/shred the treated waste to produce shredded waste. A second compartment is disposed downstream of the grinder to receive and capture the shredded waste from the grinder for removal from at least one egress.

In another aspect of the invention, a method of treating medical waste includes obtaining the aforementioned medical waste sterilization and processing device; introducing medical waste to the first sealable compartment via the top lid; sealing the first sealable compartment; and treating the medical waste by exposing the medical waste to high temperature steam from about 110° C. to about 150° C. and pressure from about 15 psi to about 60 psi for at least about 3 minutes. The bottom lid is then moved to its open position, and the press plate is moved in the downstream direction to push the treated waste out of the first compartment into the grinder, for grinding/shredding to produce shredded waste. The shredded waste is moved to the second compartment from which it may be removed from the device.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

The present invention is illustrated by way of example and not limitation in the figures of the accompanying drawings, in which like references indicate similar elements and in which:

FIG. 1A is a perspective front view of one embodiment of a device of the present invention, with doors/lids in their open positions;

FIG. 1B is a view similar to that of FIG. 1A, with portions broken away to reveal internal components, with the doors/lids in their closed positions;

FIG. 2 is an image of a dashboard generated by the embodiment of FIGS. 1A & 1B;

FIG. 3 is a perspective view of a component of the embodiment of FIGS. 1A-2;

FIG. 4 is a sectional view taken along 4-4 of FIG. 1B;

FIG. 5A is a view, on an enlarged scale, of a portion of FIG. 4;

FIG. 5B is a view, on a further enlarged scale, of a portion of FIG. 5A;

FIG. 6 is a perspective view of a component of the embodiment of FIGS. 1A-5A;

FIG. 7 is a sectional view taken along 7-7 of FIG. 1B;

FIG. 8A is a perspective view of components of the embodiment of FIGS. 1A-7, with some components in a first position;

FIG. 8B is a perspective view of components of the embodiment of FIGS. 1A-7, with some components in a second position;

FIG. 9A is a top view of components of the embodiment of FIGS. 1A-8B;

FIG. 9B is a schematic cross-section taken along 9B-9B of FIG. 9A;

FIG. 10 a view similar to FIG. 4, on an enlarged scale;

FIG. 11A is a flow chart illustrating aspects of the embodiments of FIGS. 1A-10;

FIG. 11B is a listing of data captured during operation of the embodiments of FIGS. 1A-11A; and

FIG. 12 is a block diagram of one embodiment of a computer system useful in embodiments of the present invention.

### DETAILED DESCRIPTION

In the following detailed description, reference is made to the accompanying drawings that form a part hereof, and in which is shown by way of illustration, specific embodiments in which the invention may be practiced. These embodiments are described in sufficient detail to enable those skilled in the art to practice the invention, and it is to be understood that other embodiments may be utilized. It is also to be understood that structural, procedural and system changes may be made without departing from the spirit and scope of the present invention. In addition, well-known structures, circuits and techniques have not been shown in detail in order not to obscure the understanding of this description. The following detailed description is, therefore, not to be taken in a limiting sense, and the scope of the present invention is defined by the appended claims and their equivalents.

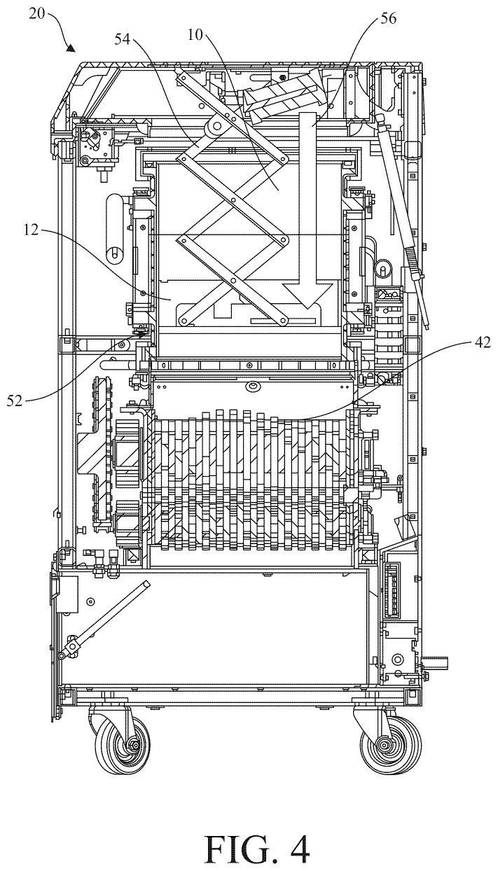

### Terminology

Where used in this disclosure, the term “axial” when used in connection with an element described herein, refers to a direction relative to the element, which is substantially parallel to a direction of travel 56 of waste through system 20 as shown in FIG. 4. Similarly, the term “transverse” refers to a direction other than substantially parallel to the axial direction. The terms “transverse cross-section” or “transverse circumference” shall refer to a cross-section or circumference, respectively, taken along a transverse plane. Similarly, the terms “upper” or “upstream” and “lower” or “downstream” refer to directions along direction of travel 56.

As used herein, the terms “computer”, “programmable logic system” and “end-user device” are meant to encompass a workstation, personal computer, tablet, wireless telephone, or any other suitable computing device including a processor, a computer readable medium upon which computer readable program code (including instructions and/or data) may be disposed, and a user interface. Terms such as “server”, “application”, “engine”, “component”, “module”, “control components/devices”, “messenger component or service,” and the like are intended to refer to a computer-related entity, including hardware or a combination of hardware and, software. Moreover, the various computer-related entities may be localized on one computer and/or distributed between two or more computers. The terms “real-time” and “on-demand” refer to sensing and responding to external events nearly simultaneously (e.g., within milliseconds or microseconds) with their occurrence, or without intentional delay, given the processing limitations of the system and the time required to accurately respond to the inputs.

As used in the specification and in the appended claims, the singular forms “a”, “an”, and “the” include plural referents unless the context clearly indicates otherwise. For example, reference to “an analyzer” includes a plurality of such analyzers. In another example, reference to “an analysis” includes a plurality of such analyses.

As used herein the term “sterilization” refers to the process of eliminating, or reducing to an acceptable level, infectious materials such as germs, noxious materials, bacteria, viruses and the like, or potentially infectious materials such as blood and other bodily fluids, rendering them harmless. The term “installation” refers to permanent or semi-permanent fixtures which require placing devices into position and connecting. The term does not refer to simple plug and unplug of an electrical cord into an electrical outlet. The term “medical waste” refers to regulated medical waste, biohazardous waste and potentially infectious waste. It does not relate to pathological waste or chemotherapy, chemical, or hazardous waste.

As used herein the term lid refers to a removable cover, positioned either on the top/upstream end, or on the bottom/downstream end, of a structure. The instant disclosure is intended for substantially all medical waste which requires sterilization to be rendered harmless, including human and animal waste as well as other biological materials found in laboratories or areas where work on biologically active materials occurs. The terms “grinder”, “shredder”, “grinding” and “shredding” refer to devices and processes that transform the medical waste into an unrecognizable form through the actions of cutting, slicing, chopping, pulverizing and the like and are used interchangeably herein.

Although specific terms are employed herein, they are used in a generic and descriptive sense only and not for purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning as commonly understood by a person having ordinary skill in the art to which this invention belongs. It will be further understood that terms, such as those defined in commonly used dictionaries, should be interpreted as having a meaning that is consistent with their meaning in the context of the relevant art and the present disclosure. Such commonly used terms will not be interpreted in an idealized or overly formal sense unless the disclosure herein expressly so defines otherwise.

### General Overview

Embodiments of the present invention represent improvements to the Sterilis Solutions Remediator, a medical waste processor commercially available from Sterilis Solutions of Boxborough, MA, that processes most forms of Regulated Medical Waste (RMW) by exposing the load to high-temperature steam to kill pathogens, followed by a grinding process that renders the entire waste load unrecognizable and reduces the waste volume by up to 80%.

These embodiments are intended for all medical waste that requires sterilization to be rendered harmless, including human and animal waste as well as other biological materials found in laboratories or area where work on biologically active materials occurs. These embodiments thus include medical waste sterilization and processing devices having a housing, lids, compartments, grinders, platforms, and other components. They may be made from any structural material known in the art, e.g., high impact plastic such as high impact polystyrene, or metals such as various types of stainless steel or aluminum. Suitable materials are readily cleanable and do not support the growth or survival of the materials to be sterilized.

The housing is fitted with an ingress allowing materials for treatment to be placed into the device. The ingress may be a sliding door, a lid, or other covered opening and may be provided with a lock, a logging device to measure when materials have been added to the device, or other mechanism to secure the ingress. The ingress may be situated on the top, the side or other area on the device to provide for convenient operation.

Exemplary devices include a first sealable compartment to which the ingress is associated, such that waste materials to be treated are placed in the first compartment. The compartment may be constructed of any structural material that can withstand the operational temperatures and pressures of the device. In some embodiments the waste materials reside in a bag specially designed for holding such waste, sometimes called a Red Bag. When the waste bag is input, the bottom is closed to accept the waste, after which the top lid is closed and both lids are secured and the first compartment is sealed. The lids use typical components of gaskets, locks and devices for high pressure sealing well known in the art, so that when high temperature steam is introduced, it is contained within the compartment during the sterilization operation. The compartment is also fitted with sensors to determine time, pressure, temperature, and duration of the sterilization processes, which may interface with a programmable logic system.

In some embodiments the top lid slides horizontally, or may pivot vertically, to open and close, to allow access to the first compartment. The bottom lid may contain a perforated platform as a top portion of the lid and is positioned above the steam generating component which is situated in the bottom portion of the bottom lid. Alternatively, the bottom lid may be communicably coupled to a steam autoclave system that generates and supplies steam to the first compartment. The perforated platform supports the medical waste to be treated. The perforated platform may be a screen, perforated metal, perforated plastic, or the like containing round holes, squares holes, slots and the like. The platform provides structural integrity to hold the waste, a bag containing waste or a combination, a waste holder, and the like and allow steam to flow through.

In some cases the air is significantly evacuated from the first compartment prior to steam generation. When the steam is generated, it flows through the perforations in the perforated platform and permeates the medical waste which is supported thereon. In typical devices, when steam is initially introduced into the first compartment, the compartment is initially cool, so that the introduced steam condenses on the wall of the compartment and collects at the bottom of the compartment, and in and on the medical waste that is to be treated. The steam then must not only bring the compartment to temperature, but heat the condensed water to become steam in order to properly sterilize the waste. These processes require much more steam than would normally be needed to sterilize a specific amount of waste. Some devices address this issue by requiring the compartment to contain a steam jacket in which steam heats the inside walls of the compartment so that the steam introduced into the compartment is less likely to condense. This again requires a relatively large amount of steam to accomplish. Generally a waste treatment device which is permanently installed can be connected to a large, dedicated steam generator connected to the device through a series of high pressure steam pipes. Other devices that have addressed steam condensation require the walls of the first compartment to be heated with electricity. Depending on the size of the device, this may be a costly solution requiring a complicated compartment configuration as well as causing high energy usage. Particular embodiments hereof address condensation by collecting any condensed water onto the heating plate of the bottom lid. When the steam condenses on the walls and collects onto the heated plate, the heated plate reheats the collected water, becoming steam again, which is then recycled into the compartment thus continuing sterilization, allowing for more efficient energy and resource usage. In this manner any steam that enters the compartment will continually be used to sterilize the waste.

Water may initially be introduced onto the heating plate from a water reservoir of a water reclamation system. After the sterilization the first sealable compartment is depressurized and the steam is directed through a filtering system and condensed back into the water reservoir. Water remaining on the heating plate is drained and returned to the reservoir through a filtration system. The reservoir is connected to an inlet which is used to replace water that has been removed with the treated waste when water has reached a low level requiring replenishment. The filter systems used are biofilters, charcoal filter, particle filters and the like, as are well known in the industry for filtering gases and liquids.

After sterilization and removal of residual steam and water the bottom lid can slide laterally while the treated waste, or waste bag, drops into the grinder positioned beneath the first sealable compartment.

The platform may pivotally turn to allow the waste to fall into the grinder or the platform may slide out of the compartment with or without a scraper that scrapes the waste off the platform to fall into grinder. The platform may also be attached to one side of the wall which falls away to allow the waste to proceed to the grinder.

Since the treated waste is typically gravity fed into the grinders, some treated waste may not be heavy enough to fall into the grinder. Embodiments hereof may further contain a component which helps the treated waste proceed through to the grinder. The component may be positioned above the top lid of the first sealable compartment and comprises a top portion, a bottom portion (press plate) and an actuator between the portions. The press plate is configured to fit into the central chamber of the first compartment. The actuator may be a scissor jack, an inflatable bladder, a piston, or other device that can push the press plate through the central chamber and help convey the treated waste to the grinders.

These embodiments include grinders and/or shredders which grind the treated waste into small, unrecognizable pieces and is positioned after the first sealable compartment. The grinder may incorporate a planetary gear box which drive a cluster of rotating cutters. The grinding mechanism may be a two stage process wherein the cutting mechanism employs a helical design with helical cutting edges. The grinder may take on any number of configurations including containing a pair of counter-rotating shafts with a plurality of cutter blades or blade knives along the length of the shafts, and a grating plate in which the blade knives rotate, and is not restricted to the kind or type of grinder, or shredder, used. Grinders and shredders suitable for devices of the current disclosure include those well known in the art for grinding medical waste into an unrecognizable form, such as, for example, those disclosed in U.S. Pat. No. 7,195,743 to Butler, incorporated herein by reference, limited to what is disclosed for grinding and shredding of medical waste.

The steam generator component of these embodiments may be contained in the bottom lid of the first sealable compartment and is configured to provide steam to the sealable compartment during operation. The steam generating component contains a heating platform and heating elements that are heated by electrical power supplied from a wall outlet and the steam that is generated is conducted through the perforations of the perforated platform to the first sealable compartment. The steam generator component also contains a water reservoir configured to supply water to the steam generator and receive water from the water reclamation system. In some embodiments there may be water supply pipes connected to the device to supply external water that feeds the steam generating component, such pipes and connections requiring installation and deinstallation when portability is desired. Other embodiments may not contain any water supply lines and may be completely self-contained. The removal of the need for installation and deinstallation allows for increased freedom of mobility of the disclosed device not available in other waste treatment devices. The water reservoir may be filled periodically by opening an inlet into the reservoir and filling it to a desired level, determined by the amount of medical waste processed, the efficiency of recycling the excessive water from the treated material and the acceptable amount of water contained in the packaged material.

A water reclamation system may be positioned between the first sealable compartment and the water reservoir. It is configured to substantially remove excess moisture from the first sealable compartment prior to further processing. The first compartment may be heated using electric power to evaporate the excess moisture, the moisture being conducted to a condenser for collection and return to the reservoir. Fans and vacuum may be used to help evacuate moisture from the compartment and/or direct it to water reclamation components such as, for example, condensers cooled by water directed from and returned to the water reservoir. The water reclamation system further may contain purification components such as, for example, HEPA filter, activated carbon filters and the like, situated prior to the water entering the water reservoir. As added precautions, other components may be present to ensure that water returning to the boiler is highly purified, such as, for example, treatment with UVC light radiation and/or ozone.

In this way the steam generating component continually is supplied with water for making steam. The temperature of the steam for sterilization of medical waste is between about 110° C. to about 150° C. and the pressure is between about 15 to about 60 psi. Depending on the amount of waste to be treated and the local regulatory requirements, the time of treatment may run from a minimum of 3 minutes once the waste load has reached 132° C. up to a minimum of 15 minutes at 121° C.

The device may also include a compactor for reducing the volume of the waste situated in the first sealable compartment. The compactor may be driven by a piston, a hinged plate or other compacting device driven by pneumatics, hydraulics or other forms of force.

Particular embodiments may also contain a second compartment which receives the treated and ground waste from the grinder. The treated waste may be moved to the second compartment by a number of methods including gravity feed or a component designed for helping to convey the treated waste to the grinders, as described supra. In particular embodiments the second compartment may be further configured to package the treated waste. There may be provided a bagging system which accepts the treated waste when moved from the grinder. The bags are designed to withstand the system's heat processes, and may be fabricated from materials such as, for example, high density polyethylene, polypropylene or other polyolefin, polystyrene, PET, and the like. When the waste is placed in the bag, the bag may optionally be sealed, heat sealed, or closed in any fashion known in the art. Substantially all the air may optionally be removed from the bag using a vacuum pump prior to sealing the bag. The bags may contain RFID tags which allow for unique identification of the bag with any number of desired information including, for example the waste producer, the amount of the waste, the levels of treatment, the point of origin, waste types, and the like. The bags may alternatively contain other indicia designed to uniquely identify the bag. The tags may be inherent to the bag or may be added to the bag during processing depending on the desired information needed on the tag. The packaged waste may be removed from an egress positioned in the front, side or back of the device as desired.

The second compartment, as well as the waste bags, may be connected to the water reclamation system so that excessive moisture may be reclaimed at any stage of the process. In some embodiments of the current disclosure the devices may run on electrical power as obtained from a wall outlet. For example, in North America, northern South America and Japan, electric power is typically supplied at 100-127 V or 220 V, 50-60 Hz to a wall socket. Most of the rest of the world supplies electrical power at 200-240 V, 50-60 Hz to their wall outlets. The current devices may use a typical plug and wire that plugs into the wall and is readily removed. Thus, no special wiring designs or permanent or semi-permanent installation are required. In other embodiments, the device may be hard wired to a power supply.

Some regulations require either longer treatment times or higher temperatures of steam when steam treating the waste, or both. To provide proper treatment the devices of the current disclosure are provided with at least one programmable logic system into which required parameters may be entered, including, for example, steam temperature, steam pressure, time of exposure of the medical waste to the steam, grinding/shredding parameters, unique identifiers of the waste, including the point of origin. The programmable logic system may also record the process information for future reference. Unique identifiers, such as, for example, RFID tags, bar codes, alpha-numeric indicia, or other identifying indicia may be created and attached to the treated waste output.

A radiation detection device may also be included in the device. Waste treatment regulations require that radioactive waste be treated differently from other medical waste, for example, medical waste treated to the level of local requirements may be disposed in a land fill, while waste containing any radioactive waste is barred from such disposal. Thus the device may be used to prevent radioactive waste from being mixed with “regular” medical waste.

Various embodiments are configured for mobility. As mentioned, these embodiments may be self-contained and free of external connections. The power supplied to the devices is through a plug into a wall outlet. Wheeling components such as wheels, castors and the like are positioned on the bottom of the device so that the device can be moved from room to room or area to area as desired. The wheeling components may include braking levers which prevent the components from moving until the levers are flipped back freeing the components to allow device movement. The devices in these embodiments are designed so that only one person, of average strength and ability, can move the device. Thus the device is below a weight and dimension that allows the movement of the device by one average person.

The devices of the current disclosure may also include quality control systems which check to calibrate the device and ensure that the various components, compartments and system of the device is in the proper operating condition such that, in operation, the device will provide the required sterilization of the medical waste. The programmable logic system can signal the various components, compartments and systems to provide feedback for operation. The logic device can then signal the operator that the device is working properly or if and where in the device a problem exists.

Methods of treating medical waste in any of the embodiments, include the steps of introducing medical waste, which may or may not be contained in a medical waste treatment bag designed for such purposes such as a “Red Bag”, into the first sealable compartment of the device via an ingress, supporting the waste onto a perforated platform contained in the bottom lid, treating the medical waste by exposing the waste to high temperature steam and pressure from the steam generating component contained in the bottom lid for a desired length of time with steam at a desired temperature and pressure, the parameters of which may have been preprogrammed into the device using at least one programmable logic system. The first sealable compartment may be evacuated removing a substantial amount of moisture, capturing the moisture by condensing it and routing it back to the steam generating component, optionally compacting the waste. The moisture may proceed through one or more filter components. The bottom lid then retreats so that the waste proceeds to the next step. The top lid may then slide horizontally to allow the component situated above the first sealable compartment to activate, to push the bottom portion of the component through the central chamber and help convey any treated waste to the next step. The treated medical waste is then conducted through one or more grinder/shredders and into the second compartment.

The second compartment may be fitted with a bag into which the treated and ground waste is collected. The air may then be optionally substantially removed from the bag, the bag be sealed, and removed from the device. The bag may be tracked using an RFID tag or other indicia for collection by a solid waste handler, and may be collected as regular solid waste.

The disclosed methods may include programming the required operational parameters into the device using a programmable logic system. These methods may also include a cleansing step after the treatment of medical waste has occurred wherein the first sealable compartment is treated with high temperature steam taken from the steam generating component. The method may also include a quality control step wherein the logic system performs a series of checks to ensure the device is operating properly compared to the programmed parameters. When the device needs to be moved, the electrical cord is removed from the wall and a person wheels the device to another area and plugs the electrical cord back into an outlet in the other area.

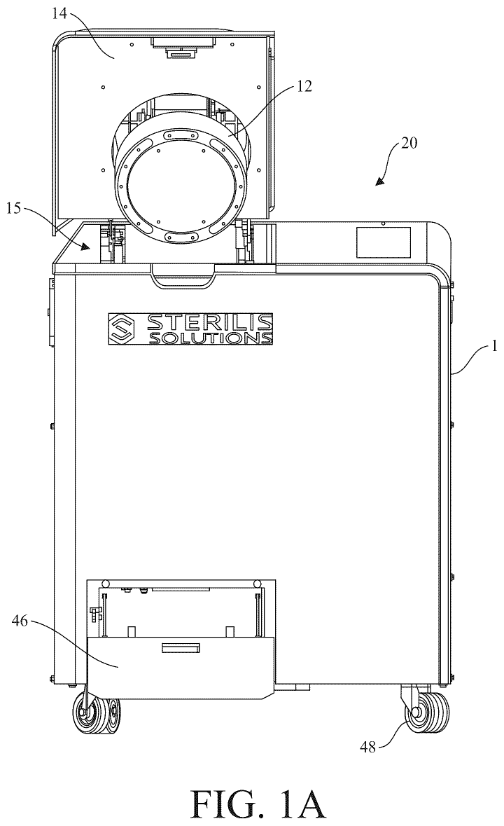

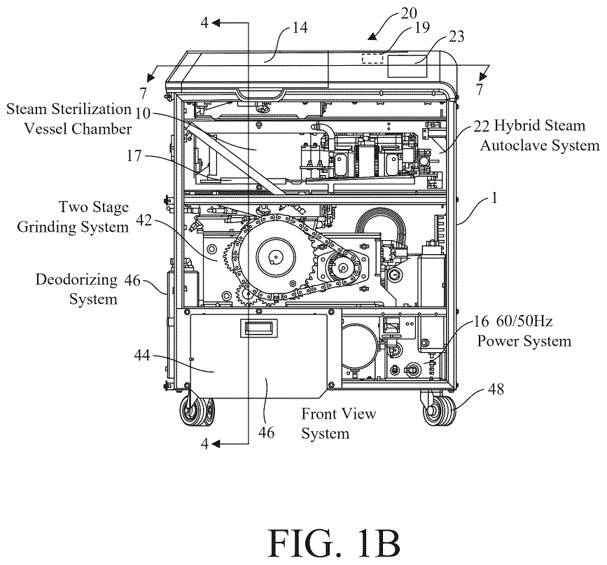

Turning now to the Figures, representative aspects of embodiments of the invention are shown and described in detail. As shown in FIGS. 1A-1B, a medical waste treatment device 20 includes a housing 1, a first sealable compartment (sterilization vessel) 10 (FIG. 1B), a conveying component (press plate) 12 (FIG. 1A), a top lid 14, and a bottom lid 17.

An inlet 15 (FIG. 1A) allows medical waste to be introduced into the compartment 10. Device 20 runs on electricity obtained from an electrical wall socket using an electrical cord from power system 16. A steam autoclave system 22 including a water reservoir supplies steam to the compartment 10. System 22 may include an evacuation pump connected to vessel 10 for drawing air and/or excess moisture out of the chamber, through a filter mechanism and back into the water reservoir. A water replenishment inlet (not shown) of system 22 is connectable to an external water supply as needed. One or more grinding/shredding devices 42 are provided between the first compartment 10 and a second compartment 44. As discussed hereinabove, second compartment 44 receives the waste after grinding/shredding. A deodorizing system 46 is optionally communicably coupled to compartment 44 to deodorize the waste. The waste is optionally collected in a bag, which is removed through egress door 46 (shown in its open position in FIG. 1A). The device 20 may be portable in which case medical grade castors 48 may be used.

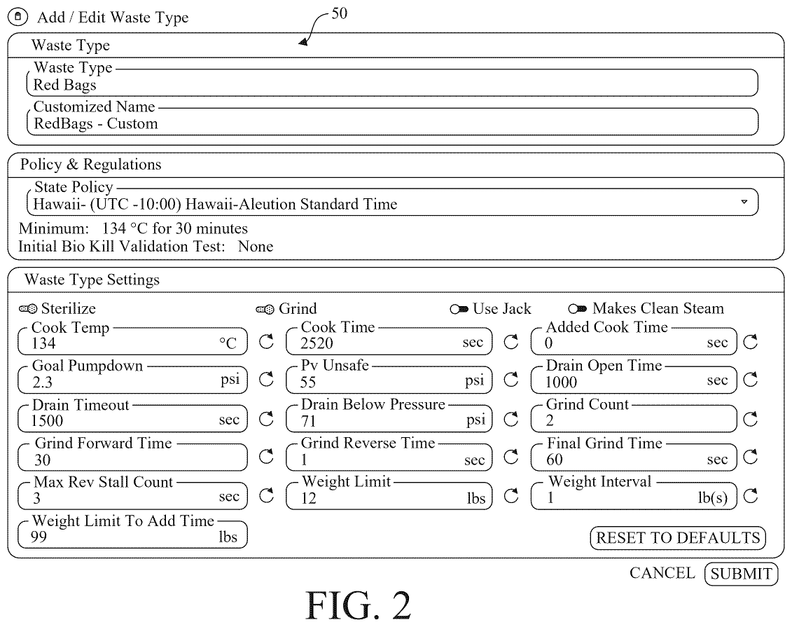

Turning now to FIGS. 2-12, various inventive improvements implemented in medical waste treatment device 20, are shown and described. These improvements include software executed by programmable logic system 19, that enables the customization of cycle parameters to meet the unique requirements of each customer/facility's waste streams proactively before installation or in response to customer requests. Authorized parties may adjust these parameters locally via a user interface displayed on screen 23, or remotely e.g., via an Internet-connected computer, e.g., to optimize waste processing and provide a customized sterilization protocol. The edited parameters may also be accessed remotely by authorized third parties for quality control, e.g., to ensure compliance with local laws and regulations, and/or to ensure validity of any required subscriptions. It is noted that these customized parameters may be optimized specifically for a particular customer/waste-generating location. Where prior approaches would only address conventional sharps containers and red bag waste methodologies for regulated medical waste, the present embodiments allow for new waste materials to be specifically dialed in for the customer's parameters and usage, including, for example, glass slides, blood tubes, plasma bags and collection containers to name a few.

For sterilization, parameters like temperature, duration at sterilization temperature, pre-sterilization vacuum pressure, allowable weight for processing, and steam drain timing may be customized. Some of these parameters, such as sterilization temperature and duration, directly impact the effectiveness of waste sterilization, requiring verification of biological kill for specific waste types at those settings. Other editable parameters, such as steam drain timing may be edited to optimize the process. For example, draining faster saves time and is optimal for a waste stream with minimal amounts of liquid. It is noted that while the sterilization principles are largely conventional, the inventors believe that the ability to provide customization by selecting different combinations of these parameters is new.

Similarly, grinding parameters may be edited, including adjustable time periods for forward grinding, reverse grinding and final grind, and activation control of a press plate that pushes waste into the grinder to detect if the process has been completed or there are additional grinding passes to be made for lighter waste materials. These parameters may enhance the grinding efficiency of different waste types. The inventors believe that the ability to change the characteristics of the grinding functions is new. While prior approaches relied on a set method for rotation and completion, the instant embodiments include new grinding parameters, such as adjusting the amount of time of the grinder runs, adjusting the usage of the forward and reversing methodologies of the grinder, using the press plate function to detect whether all materials have been shredded, and stopping the grinding function if inappropriate materials are present in the machine that may damage the grinder. For example, these embodiments allow for the independent control of Sterilization and Grinding processes, offering options such as ‘No Grind’ or ‘Grind Only’ cycles, each with specific use cases. With these editable parameters, the device permits processing multiple new waste streams efficiently with customized parameters for each facility's waste without the need to develop custom software versions.

The following is a summary of the inventive improvements to conventional approaches that are implemented in the embodiments of the present invention, and which may be actuated by the aforementioned software:
Press Plate Wiper—This feature has been added to be able to wipe away remnants in the sterilization vessel that may adhere to the sides of the chamber during sterilization. This feature was specifically added for low melting point plastics, such as collection sample bags.
Vessel Lid Wiper—Similar to the Press Plate Wiper on the inside of the sterilization vessel, this feature was added to wipe any potential remnants of waste after sterilization that may have stuck to the bottom lid surface of the system after the grinding process. This feature was specifically added to address low melting point plastics, along with polyester and vinyl stock labels that may impede the moving travel of the lids in and out of the sterilization chamber area.
Collar Blockers—Specifically added to support the software and customized waste stream creation.
Armored Seal/Gap Filler—Specifically added to support the software and the new glass slide sterilization feature.
Floating Waste Detection-Specifically added to support the software and customized waste stream creation.
Hard Object Detection—Specifically added to support the software and customized waste stream creation.
Deodorizer Spray System—Specifically added to support the software and customized waste stream creation

Notably, this improved device takes up significantly less space than other competitive platforms. Larger platforms may have the ability to process more waste, however, this device provides modularity that enables additional instruments to be connected to add capacity, while still having a smaller footprint, i.e., small enough to avoid the requirement of permits and licenses to modify the infrastructure where the systems are being installed, such as permits for water and drain attachments, or ventilation permits.

These improvements will now be discussed in detail.

As mentioned hereinabove, the software described herein enables the customization of cycle parameters to meet the unique requirements of each customer/facility's waste streams proactively before installation or in response to customer requests. Authorized parties may adjust these parameters through a web portal to optimize waste processing and to provide a customized sterilization protocol. The edited parameters may be downloaded remotely to the device for customer use and may also be reported through the portal and onto compliance reports. These waste streams become optimized specifically for that customer. Where previous approaches would only address sharps containers and red bag waste methodologies for regulated medical waste, these embodiments allow for new waste materials to be specifically dialed in for the customer's parameters and usage, including, for example: glass slides, blood tubes, plasma bags and collection containers, etc.

For sterilization, parameters such as temperature, duration at sterilization temperature, pre-sterilization vacuum pressure, allowable weight for processing, and steam drain timing may be customized. Some of these parameters, such as sterilization temperature and duration, directly impact the effectiveness of waste sterilization, requiring verification of biological kill for specific waste types at those settings. Other editable parameters, such as steam drain timing may be edited to optimize the process. For example, draining faster saves time and is optimal for a waste stream with minimal amounts of liquid.

Similarly, grinding parameters may be edited, including adjustable time periods for forward grinding, reverse grinding and final grind, and activation control of a press plate that pushes waste into the grinder to detect if the process has been completed or there are additional grinding passes to be made for lighter waste materials. These parameters may enhance the grinding efficiency of different waste types.

It is noted that the ability to change the characteristics of the grinding function is believed to be new. Prior approaches used pre-set methods of operation. In contrast, the instant embodiments provide new functionality including adjusting the amount of time of the grinder run, adjusting the forward and reversing methodologies of the grinder, use the press plate function to detect whether all materials have been shredded, and stopping and/or reversing the grinding function if inappropriate materials are present in the machine that may damage the grinder.

These embodiments also permit independent control of sterilization and grinding processes, offering options such as ‘No Grind’ or ‘Grind Only’ cycles, each with specific use cases.

With these editable parameters, these embodiments may process multiple new waste streams efficiently with customized parameters for each facility's waste without the need for developing custom software versions.

As mentioned above, these embodiments take up significantly less space than other conventional platforms. Larger platforms may be expected to have the ability to process more waste. However, these embodiments may be expanded to process larger amounts of waste, while still having a smaller footprint that may avoid the need to obtain permits and licenses to modify the infrastructure where the systems are being installed, such as permits for water and drain attachments, or ventilation permits. Additionally, by having multiple systems to process larger amounts of waste, a single point of failure is eliminated by having redundancy to process waste in multiple systems, versus having a single processing system.

Turning now to FIG. 2, an exemplary dashboard 50 generated by a processor 19 of system 20 is shown, which may be displayed on control panel 23 (FIG. 1B) and/or on a user's mobile device communicably coupled to system 20 (and programmable logic system 19) via the Internet. Dashboard 50 is a view of the customizable sterilization process that the software offers, to control various attributes discussed hereinabove. In particular embodiments, this dashboard is controlled via a remote server to create custom profiles based on local requirements, e.g., regulatory and waste content, at a particular installation.

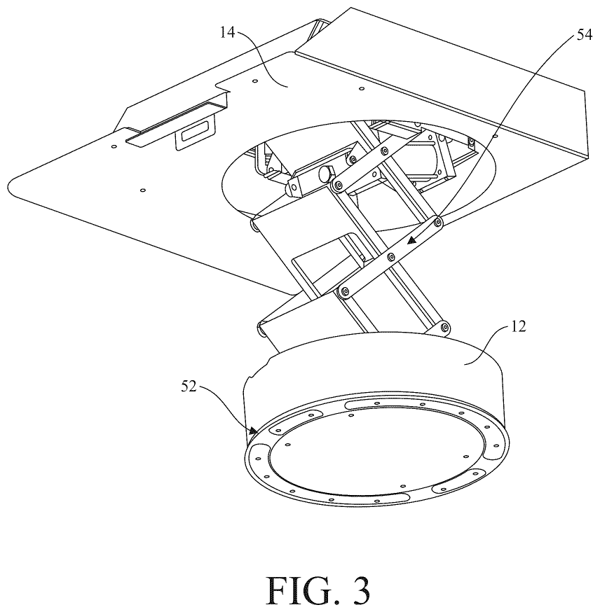

Referring now to FIGS. 3-5B, a press plate wiper 52 is disposed on press plate 12. As shown, in particular embodiments, wiper 52 is a resilient flange extending transversely to the downstream direction 56 along an outer edge/periphery of plate 12, into engagement with the inner surface of the central chamber 10. Wiper 52 is moveable in the downstream direction to wipe away remnants in the sterilization vessel chamber 10, which may otherwise adhere to the sides of the chamber during sterilization. Wiper 52 enables the system to process relatively low melting point plastics, such as collection sample bags.

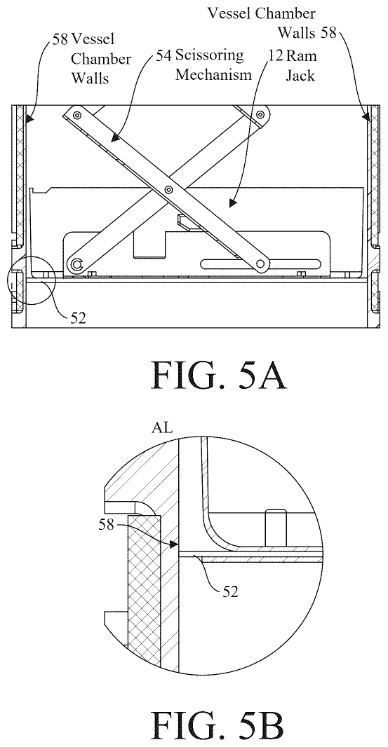

In representative embodiments, press plate 12 is moved by an actuator such as a scissoring mechanism 54 mounted to the underside of top lid 14. When lid 14 is closed (FIG. 1B), scissoring mechanism 54 may be actuated to move press plate 12 in a downstream direction through the vessel chamber 10 as shown at arrow 56, into its fully extended position as shown in FIG. 4. As best shown in FIGS. 5A & 5B, wiper 52 engages and wipes against side walls 58 of the vessel chamber 10 as the press plate 12 is actuated in the downward direction. This action effectively wipes away debris that may have attached to the side walls 58. It should be noted that wiper 52 may be fabricated from any number of materials known to those skilled in the art, in light of the instant disclosure, having sufficient stiffness to wipe the debris, while having sufficient flexibility to avoid scoring or otherwise damaging side walls 58, and while also being able to withstand the temperatures associated with steam autoclaving operations (approximately 121-134° C./250-273° F.). Exemplary materials useful for particular applications that meet the requirements of high temperature resistance (at least up to 134°C. or higher), non-abrasiveness (to avoid damaging stainless steel or aluminum walls of vessel chamber 10), chemically inert to be compatible with steam sterilization, and sufficient stiffness to scrape off debris without flexing excessively, may include: PTFE (Teflon); PEEK (Polyetheretherketone); Silicone Rubber (of relatively high durometer with reinforcement); Nylon (Heat-stabilized grades); and Fiberglass-reinforced Plastics (e.g., reinforced polypropylene or nylon).

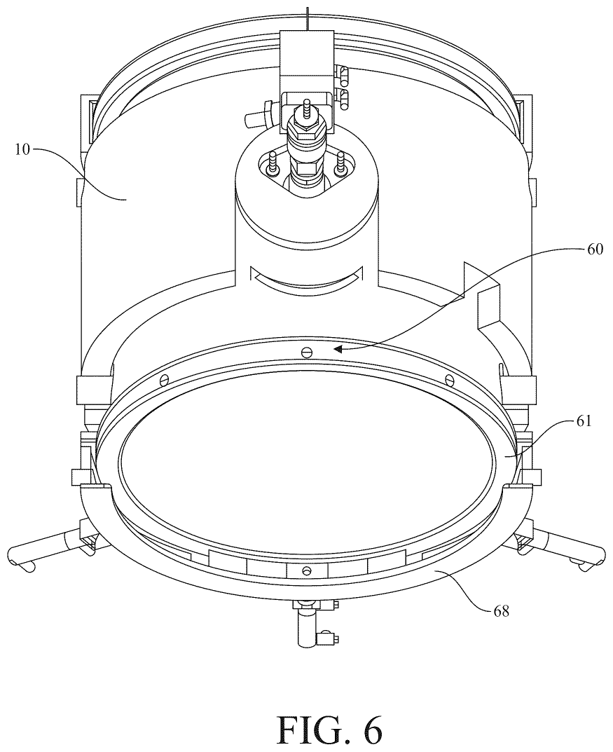

Turning now to FIGS. 6-8C, a vessel lid wiper 60, substantially similar to press plate wiper 52 but for its location, is disposed along the circumference of a lower edge portion 61 of vessel chamber 10. Wiper 60 is configured to engage an upper surface of lower lid 17 to wipe any potential remnants of waste that may have adhered to lid 17 during sterilization. Vessel lid wiper 60 is provided to address relatively low melting point plastics, along with polyester and vinyl stock labels that may otherwise impede movement of the lid 17 into and out of engagement with the lower end of vessel chamber 10 and with vessel chamber collar 68, as will be described in greater detail hereinbelow.

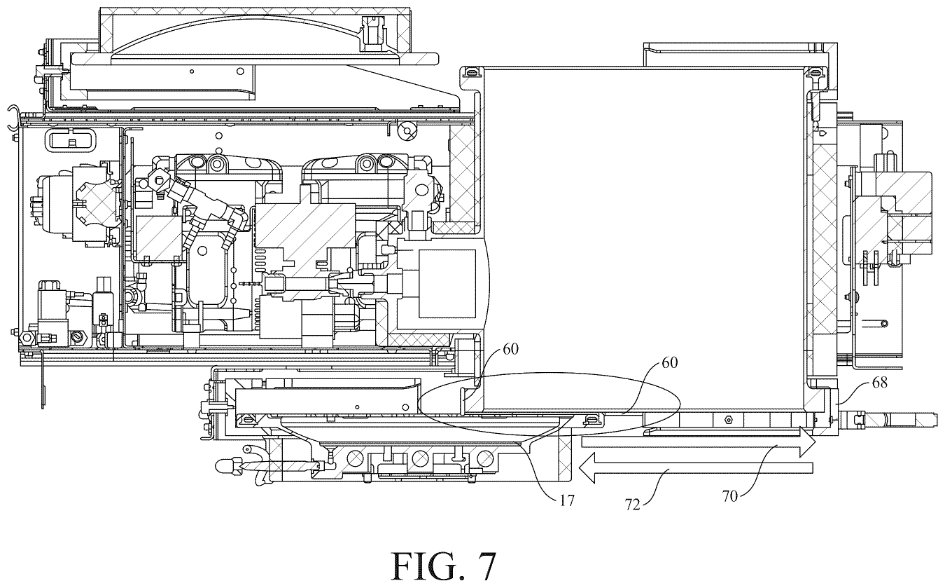

As best shown in FIG. 7, bottom lid 17 travels transversely (e.g., horizontally) back and forth to alternately open and close vessel chamber 10. Bottom lid 17 travels in the direction of arrow 70 into engagement with vessel chamber collar 68 to effectively close vessel chamber 10 for sterilization of the waste contained therein. As shown, in representative embodiments, vessel chamber collar 68 is semi-circular, extending along an approximately 180 degree portion of the circumference of the bottom of vessel chamber 10. It should be recognized, however, that collar 68 may extend less than 180 degrees without departing from the scope of the invention. Collar 68 may also be substantially straight, or provided with a curvature that is other than semi-circular, such as for use with vessel chambers that are not circular in transverse cross-section.

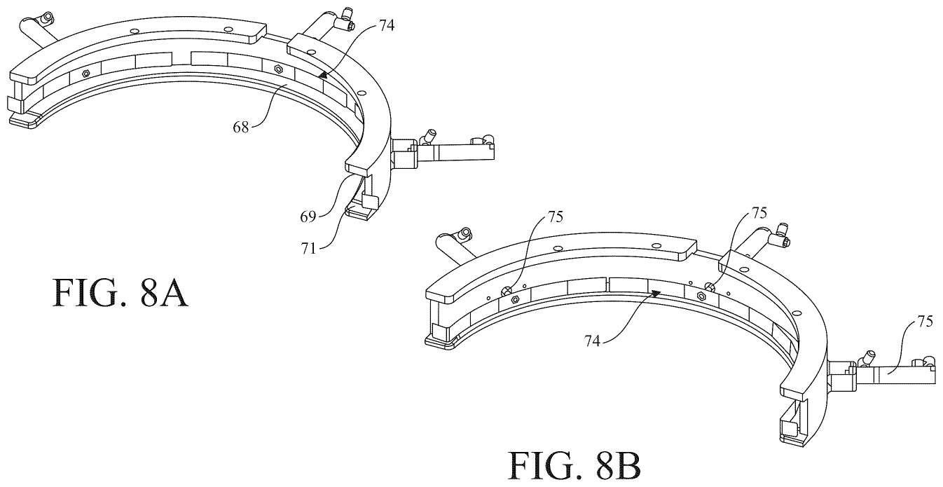

As also shown, collar 68 has upper and lower flanges 69 and 71, respectively (FIG. 8A), configured to restrict movement of chamber 10 and lower lid 17 away from one another in the axial direction when seal 82 is inflated, as discussed in hereinbelow. Upon completion

of sterilization, lid 17 travels in the direction of arrow 72 out of engagement with collar 68 to open vessel chamber 10 to permit the sterilized waste to drop into grinder(s) 42. As the bottom lid 17 travels out from under the bottom of vessel chamber 10 in direction 72 after sterilization, the upper surface of lid 17 is wiped by vessel lid wiper 62, to engage and effectively sweep any debris remaining on the surface of lid 17 into grinder 42 (FIG. 4). As the bottom lid 17 travels back in direction 70 to the closed position in engagement with collar 68, the top surface of lid 17 is again wiped by wiper 62 to sweep any remaining debris into grinders 42.

Turning now to FIGS. 8A & 8B, various embodiments include a series of collar blockers 74 extending in superposed orientation with the inner (e.g., semi-circular) surface of collar 68. Collar blockers 74 are each movable radially from retracted positions as shown in FIG. 8A, to extended positions as shown in FIG. 8B, e.g., using linear actuators 75. Once sterilization is complete, as the bottom lid 17 travels in the direction of arrow 72 (FIG. 7) out of engagement with collar 68, collar blocker 74 is moved from its retracted position (FIG. 8A) into its extended position (FIG. 8B). This action effectively sweeps any debris off of the lower flange 76 (FIG. 8A) of vessel chamber collar 68, into the shredders 42 (FIG. 4). The system moves collar blocker 74 into its retracted position when lid 17 is moved back into engagement with collar 68 to receive waste into vessel 10 and commence a new sterilization procedure. It should be noted that the various functions of system 20 as shown and described herein may be effected by the software as discussed herein.

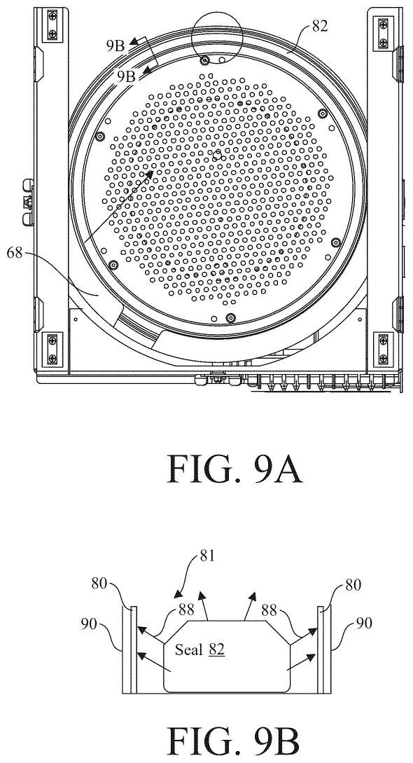

Turning now to FIGS. 9A & 9B, in particular embodiments, the upper surface of lid 17 is provided with one or more (e.g., a pair of concentric) armored seal/gap fillers 80. As shown, these seal/gap fillers are installed in an annular channel 81 on one or both sides, as shown, of an inflatable annular seal 82 used to seal lid 17 to lower edge portion 61 (FIG. 6) of vessel 10 when lid 17 is moved into engagement with collar 68. Fillers 80 are aligned with inner and outer diameters of the seal. As the seal 82 inflates during the steam sterilization process, it expands as shown at arrows 88, to engage and deflect the fillers 80 into engagement with the walls 90 of the annular groove, to create a barrier to prevent debris build up within channel 81.

Seal/gap fillers 80 are provided in particular embodiments to enable processing of glass slides and other similar medical waste that is brittle and breakable into small shards that may otherwise tend to infiltrate the seal expansion area and potentially damage or puncture seal 82. Fillers 80 may be fabricated from substantially any material capable of being deflected into engagement with walls 90 as shown, while being resistant to damage from the waste and from the heat of sterilization. In particular embodiments, fillers 80 may be fabricated from resilient stainless steel, such as 1/43011200-15 Fatigue Resistant Stainless Steel Shim Roll from Trinity Brand Industries (Burr Ridge IL. 60527 U.S.A).

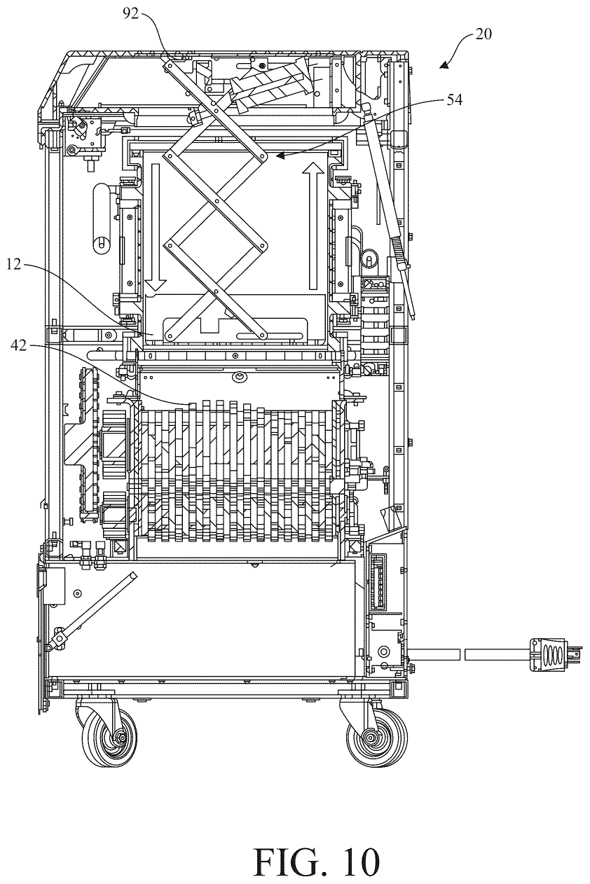

Turning to FIG. 10, in particular embodiments, the system includes a hard object/floating waste detection feature which enables customized waste stream creation for handling a relatively wide range of medical waste as described hereinabove. As shown, after sterilization and movement of lid 17 out of engagement with edge portion 61 of the vessel chamber 10 (FIGS. 6 & 7), press plate 12 is extendable down through vessel chamber 10 to push the waste into grinder 42 to shred the waste. As press plate 12 reaches the tops of the cutter blades on the grinder, an end of travel (EOT) switch 92 is engaged, indicating that there is no additional waste to shred. If EOT switch 92 is not engaged, while either a current spike, or a current drop, is observed, then the system takes action based on presence of a hard object or a floating object, respectively.

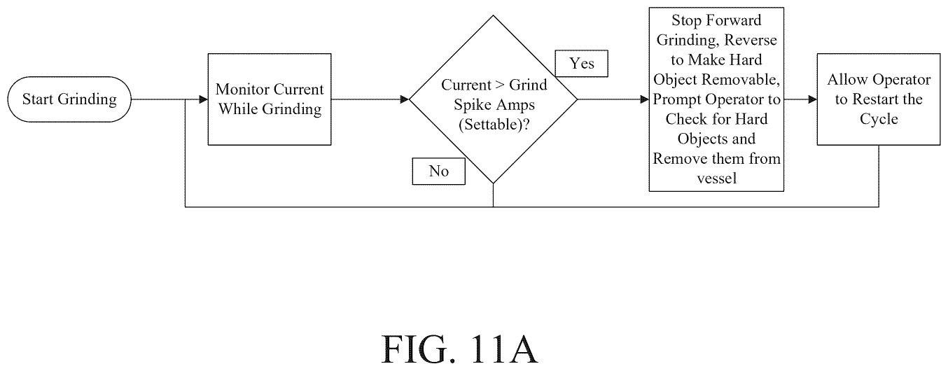

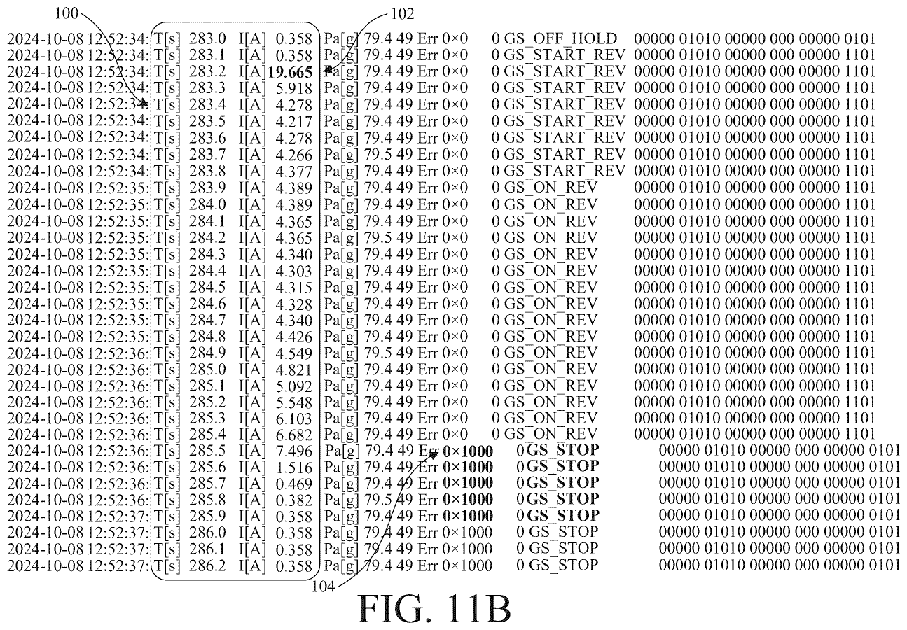

Referring now to FIGS. 11A & 11B, various embodiments are provided with a Hard Object Detection feature to support customized waste stream handling as discussed hereinabove, and to facilitate system diagnostics for preventative maintenance. As shown at 100, the operating current of the grinding system is monitored from start to finish of the grinding process, e.g., every tenth of second. The system captures the initial current before processing waste and then checks the current constantly to review the grinding process, invoking grinding stalls and reversals as harder materials are detected. In the example shown, a current spike is identified at 102 during the grinding process, and the software instructs the grinder to repeatedly reverse to try to break up the material.

For example, when a current spike 102 (FIG. 11B) is observed, which is indicative of press plate 12 encountering a hard object, the press plate retracts upwards and then moves back down in an attempt to reposition the object to be shredded. If the waste continues to be present, as indicated by EOT switch 92 being unengaged along with a current spike, then the system may alert the user to clear the object.

The software also recognizes when repeated spikes in current are returned, e.g., as shown with an Err code and STOP command to the grinder at 104. When this occurs, the grinding process is stopped, and the user is advised that there is nonregulated medical waste (Hard Object) in the system, and is instructed to remove it before completing the grinding process.

Also referring to FIG. 11B, the system provides floating object detection by capturing the initial current drawn by grinder 42 before grinding begins, and then comparing the initial current to current captured as grinding progresses. Those skilled in the art will recognize that the current will increase as the load on the grinder increases (e.g., as waste is processed by the grinder), and will decrease once the waste has been ground and there is no longer any significant load on the grinder. The system may thus use this drop in current to conclude that the grinding process has been completed. The instant inventors have recognized, however, that waste materials that have low melting temperatures, such as the labels and bags, as discussed hereinabove, have the potential to solidify and then sit or ‘float’ on top of the blades of the grinder. When this floating occurs, the current drawn by grinder 42 will drop, which may cause the system to incorrectly conclude that grinding has been completed. The instant inventors have addressed this issue by using EOT switch 92 to indicate whether the press plate 12 has reached its fully extended position at the time of current drop. In this regard, once EOT switch 92 is engaged, the system will know that press plate 12 has reached its full extension at the top of the cutter blades of grinder 42, and there is no additional waste to shred. If, however, the EOT Switch is not engaged when grinder current decreases, then the system will determine that there is floating sterilized waste in the grinder. The system may then retract press plate 12 upwards and then move it back down in an attempt to reposition the waste to be shredded. If this approach is unsuccessful and waste continues to be present and floating on the cutter blades, e.g., as indicated by the absence of any subsequent increase in current, then the system's software may alert the user to clear the floating waste.

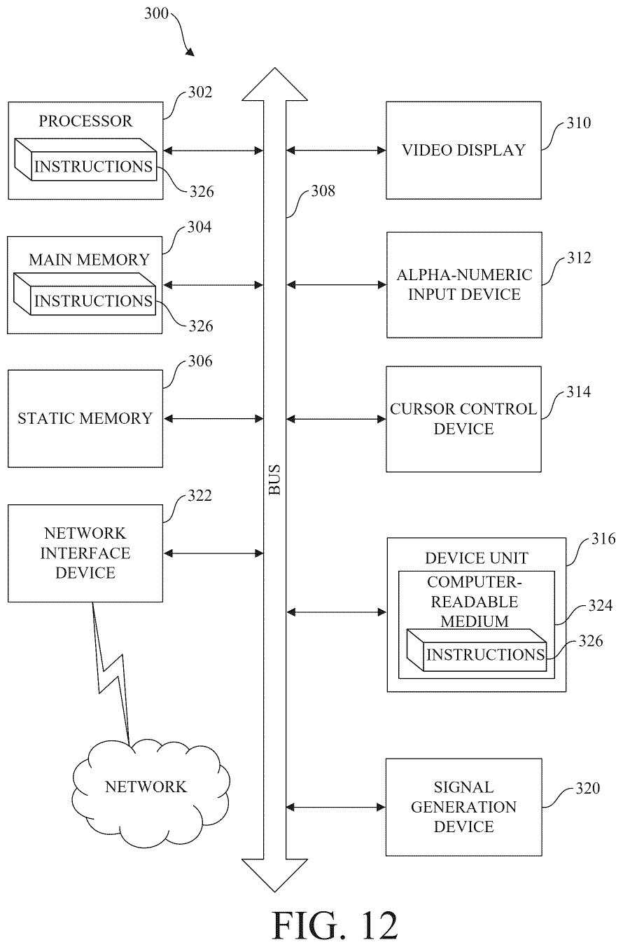

FIG. 12 shows a diagrammatic representation of a machine in the exemplary form of a computer system 300 within which a set of instructions, for causing the machine to perform any one of the methodologies discussed above, may be executed. In alternative embodiments, the machine may include a network router, a network switch, a network bridge, smartphone, tablet, a cellular telephone, a web appliance or any machine capable of executing a sequence of instructions that specify actions to be taken by that machine.

The computer system 300 includes a processor 302, a main memory 304 and a static memory 306, which communicate with each other via a bus 308. The computer system 300 may further include a video display unit 310 (e.g., a liquid crystal display (LCD), plasma, cathode ray tube (CRT), etc.). The computer system 300 may also include an alpha-numeric input device 312 (e.g., a keyboard or touchscreen), a cursor control device 314 (e.g., a mouse), a drive (e.g., disk, flash memory, etc. ,) unit 316, a signal generation device 320 (e.g., a speaker) and a network interface device 322.

The drive unit 316 includes a computer-readable medium 324 on which is stored a set of instructions (i.e., software) 326 embodying any one, or all, of the methodologies described above. The software 326 is also shown to reside, completely or at least partially, within the main memory 304 and/or within the processor 302. The software 326 may further be transmitted or received via the network interface device 322. For the purposes of this specification, the term “computer-readable medium” shall be taken to include any medium that is capable of storing or encoding a sequence of instructions for execution by the computer and that cause the computer to perform any one of the methodologies of the present invention, and as further described hereinbelow.

Some portions of above description present the features of the present invention in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times, to refer to these arrangements of operations as modules or by functional names, without loss of generality. It should be noted that the process steps and instructions of the present invention could be embodied in software, firmware or hardware, and when embodied in software, could be downloaded to reside on and be operated from different platforms used by real time network operating systems. Moreover, the particular naming of the components, capitalization of terms, the attributes, data structures, or any other programming or structural aspect is not mandatory or significant, and the mechanisms that implement the invention or its features may have different names, formats, or protocols.

The present invention is well suited to a wide variety of computer network systems over numerous topologies. Within this field, the configuration and management of large networks comprise storage devices and computers that are communicatively coupled to dissimilar computers and storage devices over a network, such as the Internet.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. It should be further understood that any of the features described with respect to one of the embodiments described herein may be similarly applied to any of the other embodiments described herein without departing from the scope of the present invention. As used in this document, “each” refers to each member of a set or each member of a subset of a set.

The present invention has been described in particular detail with respect to various possible embodiments, and those of skill in the art will appreciate that the invention may be practiced in other embodiments. First, the particular naming of the components, capitalization of terms, the attributes, data structures, or any other programming or structural aspect is not mandatory or significant, and the mechanisms that implement the invention or its features may have different names, formats, or protocols. Further, the system may be implemented via a combination of hardware and software, as described, or entirely in hardware elements. Also, the particular division of functionality between the various system components described herein is merely exemplary, and not mandatory; functions performed by a single system component may instead be performed by multiple components, and functions performed by multiple components may instead performed by a single component.

Moreover, unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Embodiments of the present invention also relate to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible, non-transitory, computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), any other appropriate static, dynamic, or volatile memory or data storage devices, or other type of media suitable for storing electronic instructions, and each coupled to a computer system bus. Furthermore, the computers referred to in the specification may include a single processor or may be architectures employing multiple processor designs for increased computing capability.

In addition, the present invention is not described with reference to any particular programming language. It is appreciated that a variety of programming languages may be used to implement the teachings of the present invention as described herein, and any references to specific languages are provided for disclosure of enablement and best mode of the present invention.

Various systems may also be used with programs in accordance with the teachings herein, or it may prove convenient to construct more specialized apparatus to perform the required method steps. The required structure for a variety of these systems will be apparent to those of skill in the art, along with equivalent variations.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

Finally, it should be noted that the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the following claims. It should be further understood that any of the features described with respect to one of the embodiments described herein may be similarly applied to any of the other embodiments described herein without departing from the scope of the present invention.

Having thus described the invention, what is claimed is:

1. A medical waste sterilization and processing device configured to move medical waste in a downstream direction therethrough, the device comprising:
a first sealable compartment having a top lid being selectively moveable between open and sealed positions, a central chamber, and a bottom lid being selectively moveable between open and sealed positions;
a steam generating component for supplying steam at elevated temperatures and pressures to the compartment;
the top lid being moveable to its open position to receive medical waste into the central chamber;
the bottom lid being moveable to its sealed position to support the medical waste within the central chamber;
the compartment configured to maintain elevated steam temperatures and pressures therein when sealed;
a press plate sized and shaped to form a sliding fit with an inner surface of the central chamber, the press plate configured for alternate upstream and downstream movement within the central chamber by an actuator;
the press plate having a resilient flange extending transversely along an outer edge thereof, to engage and wipe the inner surface of the central chamber during said upstream and downstream movement;
a grinder disposed downstream of the first sealable compartment, to receive the treated waste pushed out of the first compartment by the downstream movement of the press plate when the bottom lid is opened, and to grind/shred the treated waste to produce shredded waste; and
a second compartment disposed downstream of the grinder to receive and capture the shredded waste from the grinder for removal from at least one egress.

2. The device of claim 1, wherein the press plate is disposed on the top lid, and is configured for said upstream and downstream movement when the top lid is disposed in its sealed position.

3. The device of claim 1, wherein the bottom lid is configured for transverse movement between its open and sealed positions, to alternately open and close a downstream end portion of the sealable compartment.

4. The device of claim 3, further comprising a lid wiper extending in the downstream direction from the downstream end portion of the sealable compartment, the lid wiper configured to engage and wipe an upper surface of the bottom lid during said transverse movement.

5. The device of claim 4, further comprising a collar extending along a portion of a periphery of the downstream end portion of the sealable compartment, the collar configured to receivably engage an edge portion of the bottom lid therein when the bottom lid is disposed in its sealed position.

6. The device of claim 5, wherein the collar is semi-circular and extends along an approximately 180 degree portion of the periphery of the downstream end portion of the sealable compartment.

7. The device of claim 5, wherein the collar comprises upper and lower flanges extending transversely to the downstream direction to restrict axial movement of the chamber and lower lid relative to one another when the bottom lid is disposed in its sealed position.

8. The device of claim 7, further comprising one or more collar blockers extending in superposed orientation with an inner surface of the collar, the collar blockers each being movable radially between retracted positions and extended positions, to sweep debris off of the lower flange.

9. The device of claim 8, further comprising an inflatable annular seal disposed between the lower edge portion of the chamber and the lower lid when the lower lid is in its sealed position.

10. The device of claim 9, wherein the annular seal is disposed within an annular channel of the lower lid, and wherein at least one elongated annular seal/gap filler extends within the channel in superposed orientation with the seal, so that upon inflation, the seal expands to engage and deflect the seal/gap filler into engagement with a wall of the annular groove to effectively fill the channel and provide a barrier against debris infiltration into the channel.

11. The device of claim 1, wherein the device operates using electrical power as supplied by a wall outlet.

12. The device of claim 1, wherein the device is free of fixed attachments requiring installation.

13. The device of claim 1, wherein the device is portable.

14. The device of claim 1, further comprising a programmable logic system for user identification and monitoring, measuring, recording and analyzing the time of treatment, the steam temperature and the steam pressure for treatment of the medical waste, the weight of the waste and other user definable data and relaying the data to device controls and log files.

15. The device of claim 14, wherein the programmable logic system monitors and captures data for current drawn by the grinder during grinder operation, and uses the captured data to make adjustments to operation of the device in real-time.

16. The device of claim 15, wherein the adjustments include stopping forward grinding, starting reverse grinding, prompting a user to remove objects from the device, and combinations thereof.

17. The device of claim 1, further comprising at least one RFID identification, bar code, or identifying indicia or tracking device provided with the waste output.

18. The device of claim 1, further comprising a quality control system communicably coupled to the programmable logic system to monitor operating parameters.

19. A method of treating medical waste comprising the steps of:
a. obtaining the device of claim 1;
b. introducing the medical waste to the first sealable compartment via the top lid;
c. sealing the first sealable compartment;
d. treating the medical waste by exposing the medical waste to high temperature steam from about 110° C to about 150° C and pressure from about 15 psi to about 60 psi for at least about 3 minutes;
e. moving the bottom lid to its open position;
f. moving the press plate in the downstream direction to push the treated waste out of the first compartment into the grinder;
g. grinding/shredding the treated waste to produce shredded waste;
f. moving the shredded waste to the second compartment; and
g. removing the treated waste from the device via the second compartment.

20. The method of claim 19, wherein the device operates using electrical power as supplied by a wall outlet.

21. The method of claim 19, wherein the device is free of fixed attachments requiring installation.

22. The method of claim 19, further comprising, with a programmable logic system, identifying, monitoring, measuring, recording and analyzing the time of treatment, the steam temperature and the steam pressure for treatment of the medical waste, the weight of the waste and other user definable data and relaying the data to device controls and log files.

23. The method of claim 22, further comprising, with the programmable logic system, monitoring and capturing data for current drawn by the grinder during grinder operation, and using the captured data to make adjustments to operation of the device in real-time.

24. The method of claim 23, wherein the adjustments include stopping forward grinding, starting reverse grinding, prompting a user to remove objects from the device, and combinations thereof.

25. A medical waste sterilization and processing device configured to move medical waste in a downstream direction therethrough, the device comprising:
a first sealable compartment having a top lid being selectively moveable between open and sealed positions, a central chamber, and a bottom lid being selectively moveable between open and sealed positions;
a steam generating component for supplying steam at elevated temperatures and pressures to the compartment;
the top lid being moveable to its open position to receive medical waste into the central chamber;
the bottom lid being moveable to its sealed position to support the medical waste within the central chamber;
the compartment configured to maintain elevated steam temperatures and pressures therein when sealed;
a press plate sized and shaped to form a sliding fit with an inner surface of the central chamber, the press plate configured for alternate upstream and downstream movement within the central chamber by an actuator including a scissor jack, piston and/or an inflation bladder;
the press plate having a resilient flange extending transversely along an outer edge thereof, to engage and wipe the inner surface of the central chamber during said upstream and downstream movement;
a grinder disposed downstream of the first sealable compartment, to receive the treated waste pushed out of the first compartment by the downstream movement of the press plate when the bottom lid is opened, and to grind/shred the treated waste to produce shredded waste;
a second compartment disposed downstream of the grinder to receive and capture the shredded waste from the grinder for removal from at least one egress;
the bottom lid configured for transverse movement between its open and sealed positions, to alternately open and close a downstream end portion of the sealable compartment;
a lid wiper extending in the downstream direction from the downstream end portion of the sealable compartment, the lid wiper configured to engage and wipe an upper surface of the bottom lid during said transverse movement;
a collar extending along a portion of a periphery of the downstream end portion of the sealable compartment, the collar configured to receivably engage an edge portion of the bottom lid therein when the bottom lid is disposed in its sealed position;
the collar having upper and lower flanges extending transversely to the downstream direction to restrict axial movement of the chamber and lower lid relative to one another when the bottom lid is disposed in its sealed position;
one or more collar blockers extending in superposed orientation with an inner surface of the collar, the collar blockers each being movable radially between retracted positions and extended positions, to sweep debris off of the lower flange;
an inflatable annular seal disposed between the lower edge portion of the chamber and the lower lid when the lower lid is in its sealed position; and
the annular seal disposed within an annular channel of the lower lid, and wherein at least one elongated annular seal/gap filler extends within the channel in superposed orientation with the seal, so that upon inflation the seal expands to engage and deflect the seal/gap filler into engagement with a wall of the annular groove to effectively fill the channel and provide a barrier against debris infiltration into the channel.
